(19) United States
(12) Patent Application Publication
(10) Pub. No.: US 2025/0281650 A1
(43) Pub. Date: Sep. 11, 2025

(54) MEDICAL IMAGING TECHNIQUE USING X-RAY TO NEAR-INFRARED DOWNCONVERTING NANOPOWDER

(71) Applicant: THE TRUSTEES OF DARTMOUTH COLLEGE, Hanover, NH (US)

(72) Inventors: Xiaoxin Wang, Hanover, NH (US); Jifeng Liu, Hanover, NH (US); Shudong Jiang, Hanover, NH (US)

(21) Appl. No.: 19/217,601

(22) Filed: May 23, 2025

Related U.S. Application Data
(63) Continuation of application No. 17/322,577, filed on May 17, 2021, now Pat. No. 12,337,041.
(60) Provisional application No. 63/025,853, filed on May 15, 2020.

(51) Int. Cl. A61K 49/00; A61B 5/00

(52) U.S. Cl. CPC A61K 49/0065; A61B 5/0073

(57) ABSTRACT

A phosphor excitable by X-ray and blue-light emits light in the near-infrared (NIR-II, 1000-1700 nanometers) forms nanoparticles less than 200 nanometers diameter. The nanoparticles are tagged by coating with silica, then conjugating with polyethylene glycol (PEG) and tissue-selective compounds such as antibodies, nucleic acid chains, and other ligands. In embodiments, we administer the tagged nanoparticles to a subject, then localize the nanoparticles, and thus antigen-bearing tissues, by irradiating the subject with X-ray or other radiation beams while imaging near infrared light emitted from the subject. The nanoparticles are made by mixing 1-50 micron calcium oxide and germanium oxide powders with dilute nitric acid, adding chromium (III) nitrate at a ratio to germanium between 0.001 and 0.1, adding tartaric acid solution with molar ratio to metal ions between 1˜10, and adjusting pH to 0.1-4 with nitric acid, then later heating to form a sol, oven drying, and calcinating the sol.

### CROSS-REFERENCE TO RELATED APPLICATIONS

This application is a Continuation of U.S. application Ser. No. 17/322,577 filed May 17, 2021, which claims the benefit of priority from U.S. Patent Application No. 63/025,853, filed May 15, 2020, both of which are incorporated herein by reference in their entirety.

### BACKGROUND

Magnetic nanoparticles have been tagged with antibodies and other tissue-selective compounds. When these nanoparticles are administered to a subject, they may localize in particular tissues, such as cancer tissues, of interest to physicians. These magnetic nanoparticles can then be located to an extent by scanning the subject with magnetic fields and using specialized electronics. Resulting images are imperfect but can be useful in locating and evaluating extent of the tissues in which the nanoparticles localize.

Near-infrared (NIR) light penetrates living mammalian tissue much more readily with less scattering and absorption than do shorter wavelengths of light, such as blue visible light. Therefore, NIR bioimaging offers the advantages of deep tissue penetration, high temporal and spatial resolution, and minimal autofluorecence.

### SUMMARY

We developed a phosphor excitable by X-ray and blue-light electromagnetic radiation that, when excited, emits light in the near-infrared (NIR-II, 1000-1700 nanometer (nm)) range.

In embodiments we have formed the phosphor into nanoparticles of size 5-200 nanometers diameter, the nanoparticles of this phosphor are also known herein as the nanophosphor.

In embodiments we have formed tagged nanoparticles by coating the phosphor-containing 5-200 nanometer diameter nanoparticles first with silica, then conjugating the silica-coated phosphor-containing nanoparticles with polyethylene glycol (PEG) and tissue-selective compounds including antibodies, nucleic acid chains, and other ligands.

In embodiments, we propose administering the tagged nanoparticles to a subject, then locating the nanoparticles in the subject by irradiating the subject with focused X-ray or other radiation beams while imaging near infrared light emitted from the subject.

### BRIEF DESCRIPTION OF THE FIGURES

FIG. 1 is a schematic showing partial substitution of Ge⁴⁺ with Cr⁴⁺ in a Ca₂GeO₄ crystal structure of our X-ray or blue-light excited phosphor in near-infrared NIR-II spectral regime. In some embodiments, remaining Ge atoms are substituted with Si or Sn atoms. Similarly, in some embodiments the Ca atoms can be substituted with Mg, Sr, Ba, or Zn atoms.

FIG. 2 is a flowchart of the synthesis of nanopowders having the chemical formula tetravalent Cr-doped (Ca,M)ₓ(Ge,N)_{y}O_{z}:Cr⁴⁺ (M=Mg, Sr, Ba, Zn and N═Si, Sn) from an aqueous precursor solution of metals and additives of water-soluble acid; where Cr=chromium, Mg=magnesium, Sr=strontium, Ba=barium, Zn=zinc, Si=silicon, Sn=tin, O=oxygen, and Ca=calcium as known in the art of chemistry.

FIG. 3 is a flowchart of a synthesis for silica coating of the Cr⁴⁺-doped X-ray downconverting nanoparticle powders formed according to FIG. 2.

FIG. 4 is an illustration of X-ray diffraction patterns of the Ca₂GeO₄:Cr nanophosphor powders obtained at 700° C., 800° C., 850° C. and 950° C. It is a mixture of Ca₂GeO₄ crystal phase and Ca₂Ge₃O₁₁ crystal phase.

FIGS. 5A and 5B are TEM images of Ca₂GeO₄:Cr nanophosphor powders obtained at 950° C.:

FIGS. 5C and 5D are TEM images of Ca₂GeO₄:Cr nanophosphor powders obtained at 700° C.

FIGS. 6A, 6B, and 6C illustrate the optical properties of Ca₂GeO₄:0.05 Cr nanophosphor material. FIG. 6A is a room-temperature photoluminescence (PL) spectrum; FIG. 6B illustrates temperature-dependent photoluminescence spectra, and FIG. 6C illustrates normalized integrated PL intensity and PL spectral width FWHM as a function of temperature. The samples were annealed at 950° C. and under an excitation wavelength of 633 nm.

FIG. 7 illustrates X-ray diffraction patterns of the pure-phase Ca₂GeO₄:Cr nanopowder annealed at 950° C.

FIG. 8 illustrates a schematic protocol for preparation of surface modified Ca₂GeO₄:Cr nanopowder core/shell structures for optical bio-imaging diagnosis applications.

FIG. 9 illustrates a protocol for preparation of surface modified iron oxide/Ca₂GeO₄:Cr⁴⁺ nanobrids@silica structure for bimodal bioimaging diagnosis applications.

FIG. 10 illustrates a system for medical imaging using injected surface modified Ca₂GeO₄:Cr nanopowder core/shell structures with X-Ray stimulus and an infrared imaging camera.

FIG. 11 is a block diagram of a method of medical imaging using the Ca₂GeO₄:Cr phosphor nanoparticle nanopowder.

### DETAILED DESCRIPTION OF THE EMBODIMENTS

We disclose an efficient X-ray downconverter containing non-toxic, tetravalent-Cr doped Ca₂₋ₓMₓGe_{1-y}N_{y}O₄ (M=Mg, Sr, Ba, Zn and N═Si, Sn) nanophosphor that converts X-ray radiation into emission in the near-infrared II (NIR-II, 1000˜1700 nm) range for optical bio-imaging diagnosis and treatment. One example is a Ca₂GeO₄:Cr⁴⁺ nanoparticle powder synthesized with minimal organic substance, impurity-free and low-temperature methods. The small size (5˜200 nm diameter) of the nanoparticles is desirable to shorten the excretion time in bio-imaging applications. The tetravalent Cr⁴⁺ ions give a broad emission, covering most of the NIR-II window. The Ca₂GeO₄ host includes Ge—O₄ tetrahedral structures that can be excited with X-ray, ultraviolet, or visible light and then transfers the energy to Cr⁴⁺ active emission centers.

The Cr⁴⁺ ion itself can also be efficiently excited by broad-spectrum Cherenkov light emissions at wavelengths less than 900 nm induced by X-Ray radiation. The thermal release of carriers from the traps in the host generated by X-ray radiation or co-doping allows Cr⁴⁺ ions to give a persistent afterglow in the NIR-II range. The persistent luminescence after the removal of excitation source can be used to reduce interference by autofluorescence with infrared images and enhance the signal/noise ratio in optical bio-imaging. Therefore, (Ca,M)ₓ(Ge,N)_{y}O_{z}:Cr⁴⁺ (M=Mg, Sr, Ba, Zn and N═Si, Sn) is a promising X-ray, ultraviolet, and visible light downconversion to NIR-II nanophosphor usable in multimode bio-imaging applications.

The use of the NIR-II light for fluorescence bioimaging to view nanophosphor concentrations offers the advantages of deep tissue penetration, high temporal and spatial resolution, and minimal autofluorescence. NIR II phosphors excited with X-ray, or X-ray induced blue-light Cherenkov emissions, provide new opportunities in the deep-tissue, in-vivo optical bioimaging. X-ray enables unlimited penetration depth in biological tissues and finds wide applications in biomedical imaging, such as computed tomography (CT). The seamless integration of CT and X-ray downconversion NIR fluorescence imaging offers a more reliable multimode bioimaging method for precise diagnosis and surgery. Rare-earth (RE) ion-doped nanostructures have been investigated intensively in different hosts with various rare-earth dopants and co-dopants for biomedical applications. However, a major concern on RE-doped nanoprobes for clinical studies is the potential toxicity and excretion issues. Among the other groups of transition metal (TM) active ions, only Cr³⁺-doped ZnGa₂O₄ persistent luminescent nanoparticles are demonstrated for optical bioimaging with emissions at ˜700 nm, far below the wavelength of the NIR-II window.

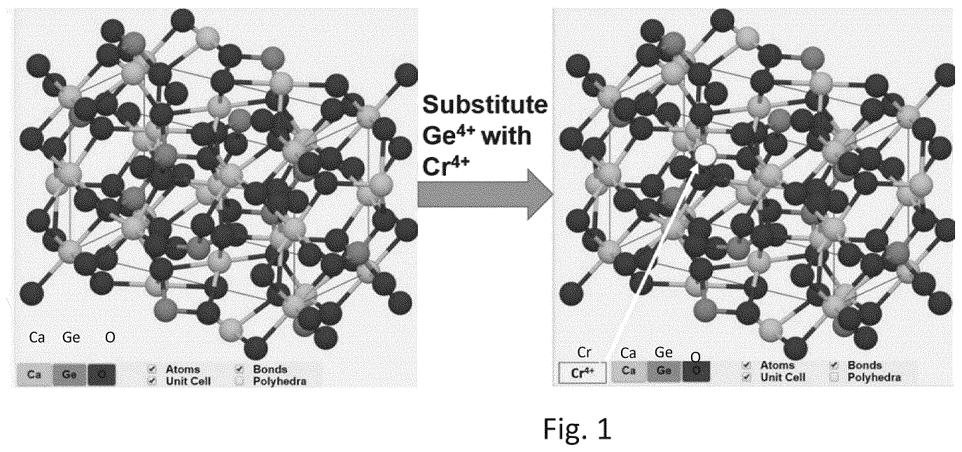

Almost all possible TM ions with different coordination numbers and configurations have been doped into single crystals and explored for tunable laser oscillation and optical amplification. A promising active TM ion is tetrahedrally coordinated Cr⁴⁺ giving emission within the NIR-II window. The broad-band emission of Cr⁴⁺ (3d² configuration) comes from the transition from the first excited state ³T₂ to the ground state ³A₂. Cr⁴⁺ ions have been incorporated in single crystals for laser oscillation and glass ceramics for optical fiber amplifiers. Considering the substitutional ion size, calcium germinate (Ca₂GeO₄) and calcium silicate (Ca₂SiO₄) are interesting hosts for Cr⁴⁺ stabilization. The similarity between ionic radii of Cr⁴⁺ (0.41 Å) and the tetrahedrally coordinated Ge⁴⁺ (0.39 Å) in Cr-doped Ca₂GeO₄ favors the substitution of the Ge⁴⁺ with Cr⁴⁺, as shown in FIG. 1. Similarly, the large difference between the ionic radius of Ca²⁺ (1.00 Å) and Cr³⁺ (0.62 Å) or Cr²⁺ (0.73 Å) inhibits the displacement of Ca²⁺ with Cr³⁺ and thus promote Cr⁴⁺ substitution of a tetrahedral Ge⁴⁺ in Ca₂GeO₄ and Si⁴⁺ sites in Ca₂SiO₄. Optical spectroscopy and EPR provide strong evidence that only Cr⁴⁺ resides in Ca₂GeO₄ and Ca₂SiO₄. Strong and high-efficiency luminescence from Cr⁴⁺ in single crystal Ca₂GeO₄ is reported. The emission intensity of Cr⁴⁺ activated Ca₂GeO₄ is one order of magnitude higher than that of Cr-doped yttrium aluminum garnet (YAG) in the similar excitation condition. CaGe₂O₄ single crystal matrix can achieve a quantum yield of 60% from Cr⁴⁺ emission at 1349 nm.

Among various valences of Cr, hexavalent Cr⁶⁺ is the ion responsible for most of the toxic reactions that may induce mutagenic and carcinogenic risks due to its solubility in water. It has also been found that the use of Cr³⁺ doped zinc gallate nanoparticles (roughly 50 nm size) brings no acute toxicity in the healthy mice. Instead, Cr³⁺ salts such as chromium polynicotinate and chromium picolinate are used as micronutrients and nutritional supplements that have demonstrated a significant number of health benefits in animals and humans. The fully stabilized and substitutional Cr⁴⁺ in Ca₂GeO₄ or Ca₂SiO₄ is not expected to impose any health toxicity. Cr⁴⁺ active ions could also be excited efficiently by X-ray and X-ray-induced Cherenkov emission. Other compounds containing Ge—O₄ tetrahedral structures have been demonstrated to absorb X-Ray and store the energy, which enables persistent afterglow and eliminate autofluorescence in the optical bio-imaging. Therefore, tetravalent-Cr doped Ca₂GeO₄ nanoparticles can be used as non-toxic, X-ray excited, NIR II phosphors.

Synthesis of Ca₂GeO₄ single crystals or ceramic/glass generally involves a calcination temperature >1200° C. with a conventional solid-state reaction approach. A sol-gel method has been used for fabrication of CaGe₂O₄:Cr⁴⁺ ceramic powder at 850° C., however, when germanium alkoxide is used as the source of Ge the alkoxide hydrolyses so fast that the control of particle size by controlling the pH of the reaction medium is not feasible. The size of CaGe₂O₄ particles synthesized by a sol-gel method starting with metal alkoxides is in the range of 500-1000 nm, which is too large for bioimaging applications. For in vivo bio-imaging applications, we prefer particle size less than 50-200 nm to reduce the excretion time. Calcination is performed in air.

To address the above-mentioned challenges, we focus on tetravalent Cr-doped (Ca,M)ₓ(Ge,N)_{y}O_{z}:Cr⁴⁺ nanophosphors with diameters <200 nanometers (nm) with less than 5% of Ge substituted with the Cr⁴⁺ for bioimaging applications. The selection criteria of calcium germinates is that Ge is in four-fold coordination (Ge—O₄ tetrahedral structures) suitable for Cr⁴⁺ substitution and stabilization, such as Ca₂GeO₄ and Ca₂Ge₃O₁₁. M represents Mg, Sr, Ba, or Zn substituting for Ca by between 0% and 100% and N represents Si or Sn substituting for Ge by 0%-100%. The partial substitution of Ca with M, and Ge with N is beneficial to enhance the emission and allows tuning of the emission wavelength of Cr⁴⁺. In embodiments, the molar concentration of Cr varies between 0.01 mol. % and 5 mol. %. A simple, low-cost, impurity-free polymeric precursor route is employed to synthesize nanophosphors consisting primarily of (Ca,M)ₓ(Ge,N)_{y}O_{z}:Cr⁴⁺ nanoparticles based on preparing an aqueous precursor solution of metals followed by adding a water-soluble L-tartaric acid as a combined chelating agent and template medium.

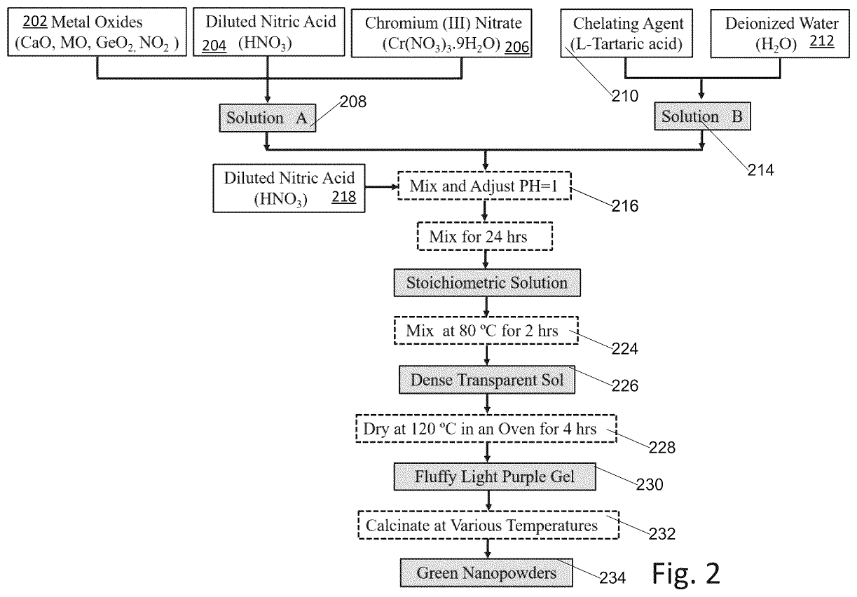

In a particular embodiment, the powders according to FIG. 2 were formed as follows:
1. Metal oxide powders 202 (1-50 microns in particle diameter) as raw materials of the host are mixed with diluted nitric acid 204 (4 wt. %˜10 wt. %), followed by the addition of chromium (III) nitrate 206 as the starting material of tetravalent Cr dopant (molar ratio of Cr to Ge is 0.001˜0.1). The formed solution is named solution A 208.
2. L-tartaric acid 210 is dissolved in deionized water 212 to form solution B 214 with the concentration range of 10 wt. %˜50 wt. %. Then two solutions are mixed with the molar ratio of L-tartaric acid in solution B to metal ions in the solution A in the range of 1˜10 and stirred for 5-24 hours at room temperature, while pH of the aqueous solution is adjusted 216 to 0.1-4 (in an embodiment 1.0) with diluted nitric acid 218 and mixed for 24 hours. The resulting stoichiometric solution is mixed 224 for another 2˜4 hours at 80° C., and in an embodiment 2 hours, to evaporate the water to form a dense transparent sol 226. The sol becomes fluffy light purple gel 230 after 4 hours drying 228 in an oven at 120° C.
3. The gel undergoes a calcination process 232 at various temperatures (700˜1000° C.) to form impurity-free green nanopowders. The calcination process involves the decomposition of carbonate and nitrate to remove other elements such as C, H and N, and heat treatment to induce the phase transformations. In this technique, uniform particle size nanopowders 234 are produced due to a homogeneous metal ion distribution in the stoichiometric solution. The synthesis process involves minimal organic substances and produces minimal polluted gas. In a particular embodiment, nanopowder of 20 nanometer average particle size is formed.

Nanophosphors face challenging issues, such as a high density of non-radiative centers due to dangling bonds or other surface states, and the change of local interaction and anisotropic degree of active ions, the preferred location of active ions at the surface. Therefore, surface passivation and co-dopants (such as Mg) are used to passivate or minimize non-radiative centers and promote the substitution with active ions, respectively.

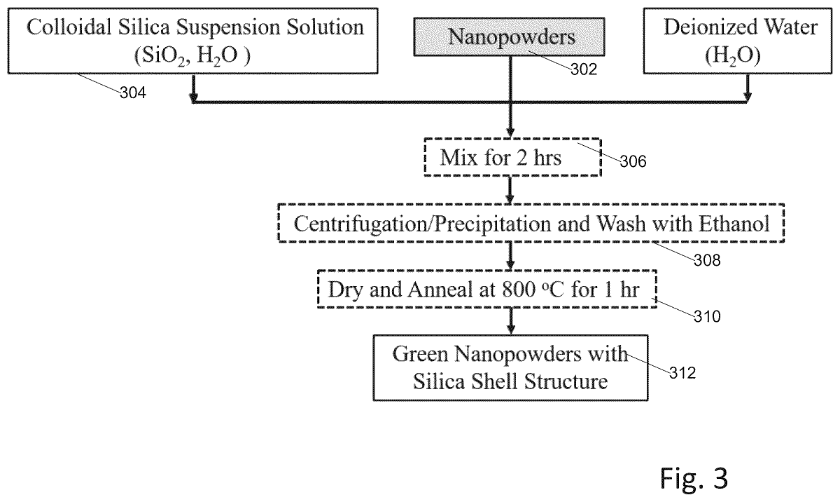

As an example of surface passivation approaches, the process flow of silica shell structure is described with reference to FIG. 3. Firstly Ca₂GeO₄:Cr nanopowder 302 (a nanopowder herein means a powder comprising primarily nanoparticles, and a nanophosphor herein means a nanopowder comprising primarily a phosphorescent material) 302 is dispersed in deionized water with weight ratio nanopowder to water of 0.001˜0.02. Then it is mixed 306 with commercial or lab-made colloidal silica suspension solution 304 (silica particle size is 5˜20 nm) with a molar ratio of silica to Ca₂GeO₄ powder in the range of 0.001˜0.1 for 1˜2 hours at room temperature. It is followed by a centrifugation/precipitation and washing procedure 308 with ethanol. The final nanopowder 312 with silica shell structure (shell thickness of 5˜50 nm) is obtained after drying and annealing 310 at 800° C. for 1 hour.

### Exemplary Systems

In the following, we give six exemplary systems for this invention. Note that the basic concept and methods are not limited to the examples given herein:

### Example 1: Ca₂GeO₄:Cr Nanophosphor Formation by the Polymerization Sol-Gel Method

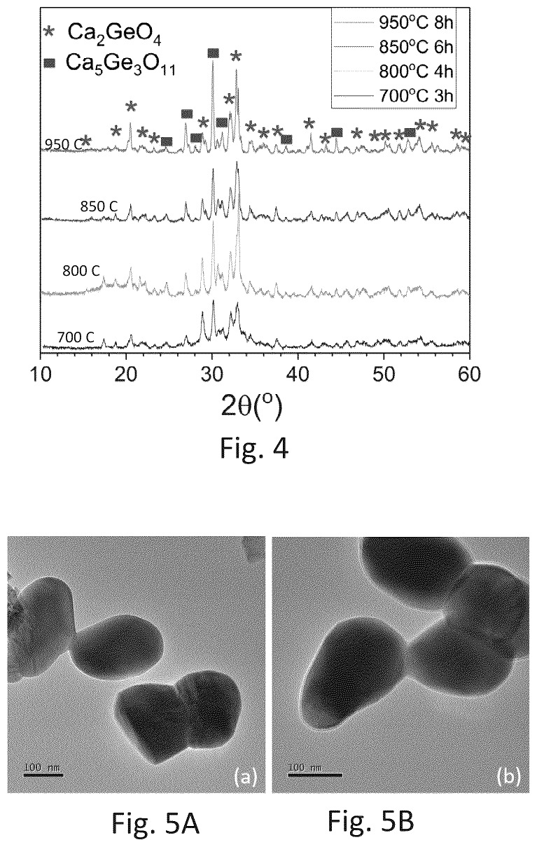

The high-purity starting materials germanium oxide (GeO₂) and calcium oxide (CaO) were weighed by the molar ratio Ca/Ge of 2.0 and then dissolved in 4 weight percent (wt. %) diluted nitric acid (HNO₃). The starting dopant material Cr(NO₃)₃ with the molar ratio Cr/Ge of 0.05 was mixed in the above-mentioned solution. L-tartaric acid was dissolved in deionized water to form a solution of 25 wt. %. The two individual solutions were mixed with the molar ratio L-tartaric acid to Ge of 4:1 under constant magnetic stirring for 24 h at room temperature. The pH of the final solution is adjusted to be 2.0 by adding more diluted HNO₃. Then the mixture was heated at 80° C. for 2 h under magnetic stirring to form a dense sol. The sol was then heated at 120° C. for 4 h and dried to form a porous purplish precursor. Finally, the precursor was annealed in the air at a temperature between 700˜950° C. at a heating rate of 1-10° C./min. The final product is fine greenish powder, indicating the conversion of Cr (III) to Cr (IV). FIG. 4 compares X-ray diffraction patterns of the Ca₂GeO₄:Cr nanophosphor powders obtained at different synthesis temperatures and duration. Most peaks are ascribed to Ca₂GeO₄ (indicated with *) and the peak broadening suggests the particle size in the nanometer range. Ca₂GeO₄ phase begin to form at a temperature of 700° C., much lower than the conventional solid-state-reaction method (1250° C.). Other crystal phase Ca₅Ge₃O₁₁ is also identified, but no raw materials (GeO₂, CaO or CaCO₃) are detected. In both Ca₂GeO₄ and Ca₅Ge₃O₁₁ hosts, Ge is in tetrahedral sites suitable for Cr⁴⁺ substitution. Differently, in the orthorhombic structure of Ca₂GeO₄, Ge has one type of tetrahedron and Ca is in an octahedral position, while in the triclinic structure of Ca₅Ge₃O₁₁, Ge has four different distorted tetrahedral and Ca is in five different octahedra. The different structures of germinate hosts can be used to engineer the Cr⁴⁺ emission (peak position, spectral width, intensity and persistent afterglow).

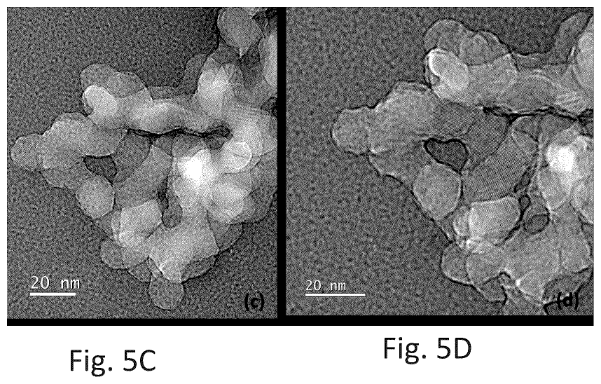

FIGS. 5A and 5B are transmission electron microscope (TEM) images of Ca₂GeO₄:Cr⁴⁺ nanoparticles obtained at 950° C., while FIGS. 5C and D are those obtained at 700° C. The average nanoparticle diameter is decreased from 100˜200 nm in the former case to 10-20 nm in the latter case. The pH value of the mixed solution and ratio of L-tartaric acid to Ge ions can be optimized to obtain the pure Ca₂GeO₄ phase and reduce the particle size to <50 nm at a lower calcination temperature such as 700-750° C.

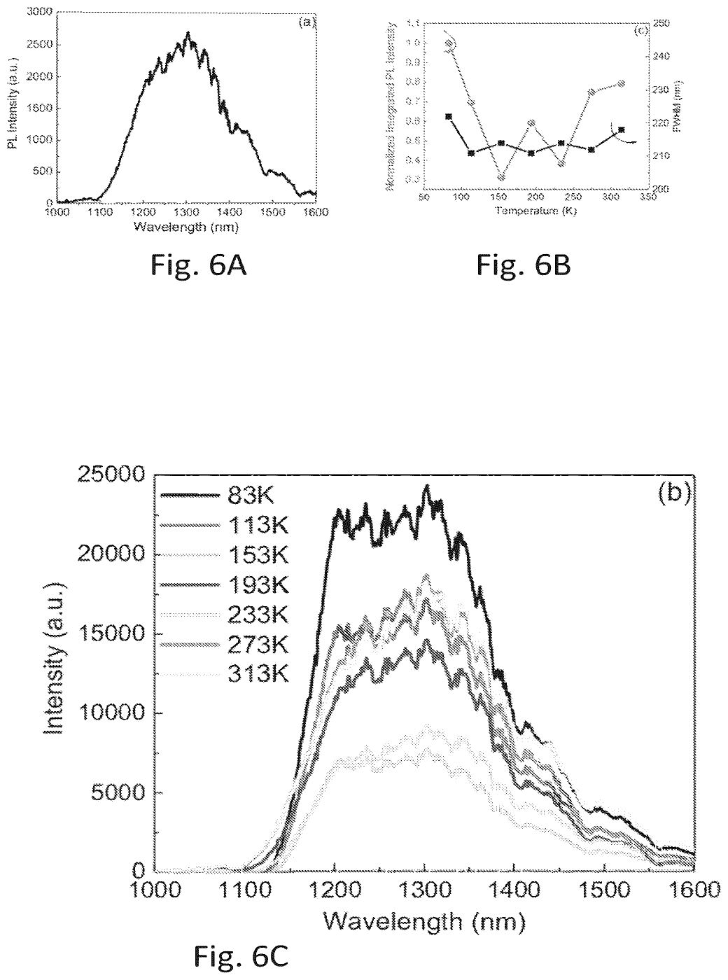

FIGS. 6A, B, and C illustrate optical properties of Ca₂GeO₄:0.05 Cr. Room-temperature photoluminescence (PL) in FIG. 6A spans from 1100 nm to 1600 nm, covering most of the NIR-II window. The peak position lies at 1300 nm, confirming that the Cr⁴⁺ emission centers are optically active. FIGS. 6B and 6C reveal the temperature-dependent PL behavior, quite different from single crystal Cr⁴⁺-doped Ca₂GeO₄. The full width half maximum (FWHM) of the PL spectra is kept at roughly 200 nm, independent of the temperature. The integrated PL intensity of nanophosphor does not follow the monotonic decrease with the increase of temperature. Instead, the integrated PL decreases first when the temperature raises from 83 K to 233 K, then gradually increases beyond the room temperature. The absence of thermal quench at body temperature (37° C. or 310K) make these nanoparticles especially attractive for in vivo applications. Some reports show the defects formed in germinate hosts (Ca₂GeO₄, Ca₂Ge₇O₁₆) can act as carrier trapping centers leading to visible persistent emission. We plan to examine the persistent afterglow performance of Ca₂GeO₄:Cr, which could extend the persistent luminescence to NIR II range. The advantage of briefly-persistent NIR-II is we can eliminate the autofluorescence and reduce the background noise in the optical bio-imaging by gated image capture in windows following pulses of the X-Ray or other radiation used to stimulate the phosphor.

### Example 2: Ca₂GeO₄:Cr⁴⁺ Nanophosphor Coated with Silica Shell

The synthesized Ca₂GeO₄:Cr nanophosphor powder of example 1 was coated with a silica shell for surface passivation. First, Ca₂GeO₄:Cr⁴⁺ powder was dispersed into colloidal silica suspension with a molar ratio of silica to Ca₂GeO₄ powder in the range of 0.001˜0.1. Then the suspension was stirred at room temperature followed by a washing procedure. The final core-shell powder is obtained after drying at 700˜900° C. The thickness and mesoporosity of the silica shell are determined by colloidal silica size and concentration. The enhancement in PL intensity and the increase in the decay time of core-shell powders can be achieved using this method.

### Example 3: Ca₅Ge₃O₁₁:Cr⁴⁺ Nanophosphor Synthesis

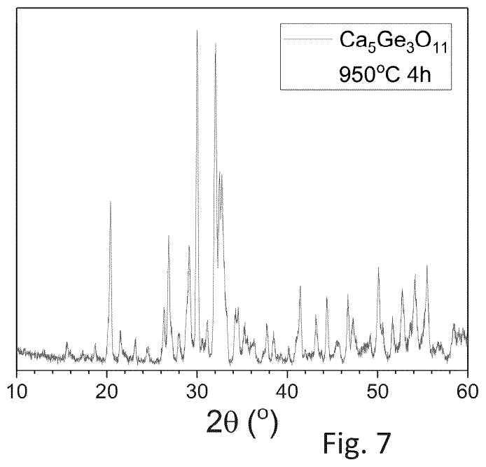

Following the similar procedure in example 1, Ca₅Ge₃O₁₁:Cr⁴⁺ pure-phase nanophosphor is synthesized with the molar ratio L-tartaric acid/Ge of 8:1 and PH of 0.3 FIG. 7 exhibits the X-ray diffraction pattern of pure Ca₅Ge₃O₁₁ phase obtained at 950° C. All peak positions in good agreement with triclinic crystal structure of Ca₅Ge₃O₁₁. The small amount of Cr⁴⁺ dopant (molar ratio of Cr to Ge of 0.01) affect the host crystal structure negligibly.

### Example 4: PEGylated Ca₂GeO₄:Cr⁴⁺ @Silica Core/Shell Nanophosphor for Optical Bioimaging

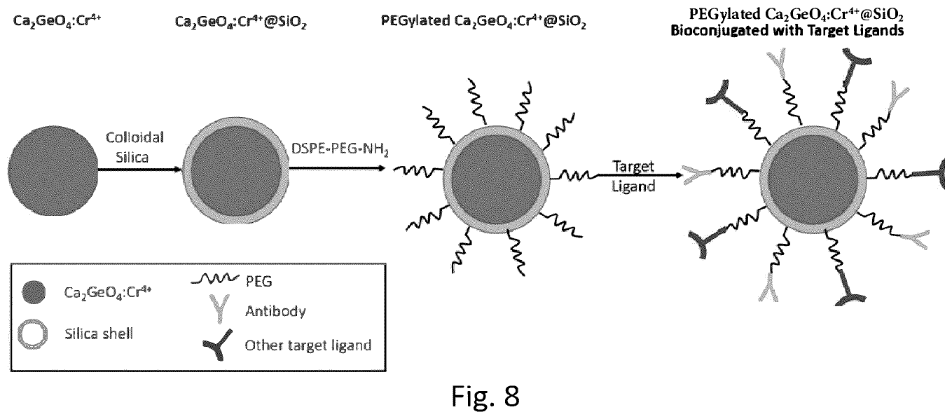

FIG. 8 illustrates a schematic protocol of nanophosphor surface modification for optical bio-imaging diagnosis application. The as-prepared Ca₂GeO₄:Cr⁴⁺ @silica core/shell nanophosphors described in Example 2 are further treated with phosphatidylethanolamine(DSPE)-polyethylene glycol(PEG)-ammonia(NH₂) to modify the surface of the nanoparticles such that it becomes a hydrophilic surface. The PEG functionalization can minimize the nonspecific interactions of nanophosphor with protein and increase the in vivo circulation time. Meanwhile, it enables nanophosphors usable in biologically relevant media and offers a platform for bioconjugation of targeting ligands, such as antibodies. To obtain hydrophilic PEGylated nanophosphor, Ca₂GeO₄:Cr⁴⁺ @silica core/shell particles are dispersed in deionized water by sonication for 3 hrs. DSPE-PEG-NH₂ or PEG is then added into the suspension mixture and stirred for 20 hrs. The weight ratio of nanophosphors, deionized water, and PEG is 1:10:10. The resulting PEG-modified nanophosphor is washed three times with deionized water to remove PEG residue and further filtered by 0.3 μm syringe filters to eliminate large particles. The PEGylated nanophosphors can be decorated with specific molecular ligands for active targeting applications by means of chemical reaction, ligand exchange, ligand-ligand interaction, etc. For example, by introducing the folate (FA) ligand to replace —NH₂ toward aqueous solution on the surface of the nanophosphors, we can facilitate the specific binding of nanophosphors to folate receptor positive tumor cells for optical targeted bioimaging diagnosis.

### Example 5 γ-Fe₂O₃/Ca₂GeO₄:Cr⁴⁺ Nanobrids for Bimodal Bioimaging Diagnosis

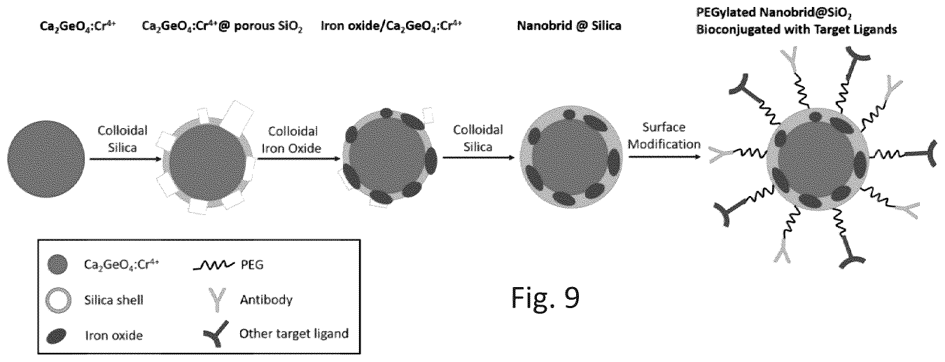

The magnetic resonance imaging (MRI) negative contrast properties provide a high spatial resolution. Bimodal bioimaging diagnosis can be achieved by combining both the advantages of MRI and optical properties of iron oxide/Ca₂GeO₄:Cr⁴⁺ nanobrids. FIG. 9 exhibits a schematic protocol for surface modified iron oxide/Ca₂GeO₄:Cr⁴⁺ nanobrids@silica. Following example 2, a mesoporous silica shell is formed around Ca₂GeO₄:Cr⁴⁺ nanophosphor by choosing the appropriate colloidal silica size and concentration. Ultrasmall superparamagnetic γ-Fe₂O₃ nanoparticles are then embedded in the mesoporous silica shell with colloidal iron oxide, similar to the method of colloidal silica. Another silica shell is coated to cover iron oxide nanoparticles. The weight ratio of iron oxide to Ca₂GeO₄:Cr⁴⁺ nanophosphor is 5%˜50%. The size of iron oxide nanoparticle is in the range of 5˜20 nm. The magnetic iron oxide labeling enables MRI tracking and targeting. Further surface modification for the optical targeted bioimaging diagnosis can be obtained on the surface iron oxide/Ca₂GeO₄:Cr⁴⁺ nanobrid@silica shell functionalized with target ligands.

### Example 6: Ca₂GeO₄:Cr⁴⁺ @Silica Core/Shell Nanophosphor for Targeted Photothermal Therapy

One example for the treatment of cancers is the targeted photothermal therapy using the heat (ideally beyond 42° C.) generated from the light radiation to destroy cancer cells. The inorganic materials for photothermal agents include metallic nanostructures (Au, Ag) and metal oxide nanoparticles (Fe₃O₄). Following the similar preparation protocol described in FIG. 9, colloidal Fe₃O₄ or Au nanoparticles are employed for conversion laser radiation to heat. Additional advantages of use of Au nanostructures is the plasmon-enhanced absorption. Meanwhile, it is possible to enhance Ca₂GeO₄:Cr⁴⁺ emission by engineering Au nanostructures.

### Application to Medical Imaging

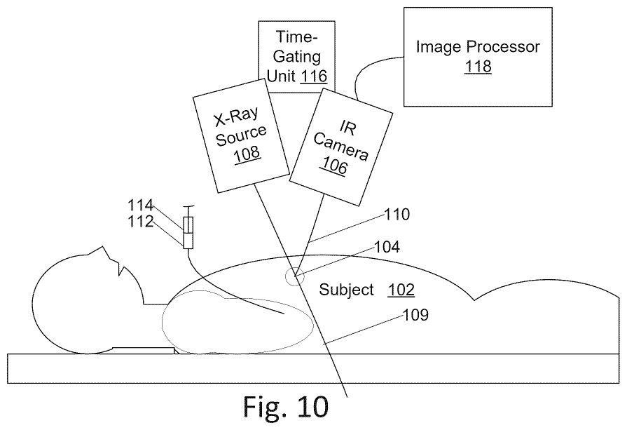

A medical imaging system for imaging in a subject 102 is illustrated in FIG. 10. The subject, having a suspect tissue such as a tumor 104, is positioned with an infrared camera 106 positioned to image tumor 104. An X-Ray radiation source 108 is positioned to provide a radiation beam 109 that intersects tumor 104. A suspension of the PEGylated Ca₂GeO₄:Cr⁴⁺ @silica core/shell nanophosphor 112 tagged with tissue-selective molecules such as antibodies, nucleic acids, or other ligands is positioned in an injection apparatus 114 and injected into subject 102. The nanoparticles with tissue-selective molecules are given time to bind with tumor 104. X-Ray source 108 is activated, providing radiation beam 109 intersecting tumor 104, causing the nanoparticles to phosphoresce, providing NIR-II light that passes through skin and other tissue of subject 102 to infrared camera 106, where the light is imaged.

In an embodiment, a time-gating control unit 116 is provided to synchronize infrared camera 106 to pulses of the X-ray beam 109. In an embodiment, time-gating control unit 116 controls infrared camera 106 such that camera 106 is time-gated to perform imaging of near infrared light immediately after, but not during, pulses of the X-ray beam 109 to reduce interference from autofluorescence of the subject's tissues. In an embodiment, a digital image processing system 118 is provided to capture and enhance the near-infrared images captured by camera 106.

In embodiments, the nanophosphor is stimulated by light from Cherenkov emissions from either an X-ray or charged-particle beam, in addition to direct stimulation from the beam itself.

In a particular embodiment, in order to provide enhanced imaging, the radiation beam is swept across tumor 104 either by sweeping a collimator at X-ray source 108 or, as in a computed tomography (CT) scanner, rotating X-ray source 108 while camera 106 is configured to capture a sequence of images of the infrared light emitted by the nanophosphor in subject 102 and bound to tumor 104. In this embodiment, the nanophosphor used has a short persistence time, 100 milliseconds or less, so that each image of the sequence of images corresponds to a particular position and/or angle of the radiation beam so that a sequence of images can be obtained using different beam positions and/or angles. In an embodiment using a rotating X-ray source 108, a reconstruction algorithm similar to those used in CT scanner is used to generate a three-dimensional image of tumor 104 and surrounding tissues in subject 102.

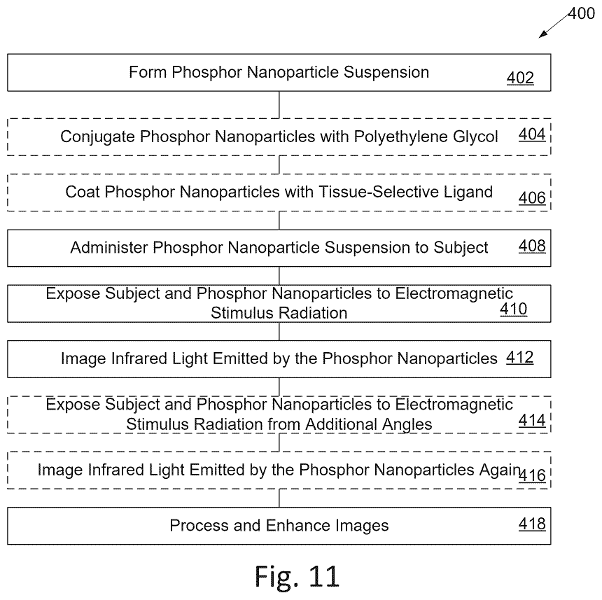

Putting the imaging method together, the method 400 (FIG. 11) of medical imaging begins with forming 402 the phosphor nanoparticle suspension. The nanoparticle suspension may be tagged by PEGylated with polyethylene glycol 404 and coated 406 with tissue-selective ligands before being administered 408 to a subject where the tagged phosphor nanoparticles may concentrate in a particular organ or tumor. The subject and tumor, with the phosphor nanoparticles, is then exposed to 410, or illuminated, with a beam of stimulus radiation that in embodiments is electromagnetic stimulus radiation consisting of X-ray radiation or blue light. In alternative embodiments the subject and tumor, with the phosphor nanoparticles, may be exposed to a particle beam that forms blue-light Cherenkov electromagnetic stimulus radiation upon interacting with tissue of the subject. Near-infrared light emitted by stimulated phosphor nanoparticles is then emitted from the subject and imaged 412 with a gated infrared camera. The subject may be illuminated with additional beams of X-ray radiation or particle beams having different beam shape or position, or beams of X-ray radiation or particle beams arriving at the subject and tumor from at least a second or additional positions or angles, to expose 414 the subject and tumor differently and additional images 416 are taken. In an exemplary embodiment, a narrow fan-beam of X-ray radiation is used and swept across the subject and tumor, the long-wavelength or infrared images of the subject in this embodiment effectively images a sequence of slices of different depths below surface of the subject and thus gives three-dimensional information regarding the subject or tumor in the subject; in another exemplary embodiment an X-ray radiation source is rotated about the subject and scanned along the subject in the manner of a CT machine, illuminating the subject and tumor with beams from multiple positions and angles while imaging is performed. The long-wavelength or infrared images may then be processed 418 to enhance them and provide significantly improved 3-dimensional images of, or a three-dimensional model of, the tumor to allow evaluation of possible treatments of the tumor.

Changes may be made in the above methods and systems without departing from the scope hereof. For example, although machine 100 is illustrated with a bevel head apparatus 125, other configurations for holding and manipulating torch 120 may be used. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A nanophosphor excitable by X-ray or blue-light radiation that, when excited, emits light in the near-infrared having wavelength between 1000-1700 nanometers consisting primarily of phosphor having the chemical formula (M)ₓ(N)_{y}O_{z}:Cr⁴⁺;
the M being one or more elements selected from the group consisting of calcium (Ca), magnesium (Mg), strontium (Sr), barium (Ba), and zinc (Zn);
the N being one or more elements selected from the group consisting of germanium (Ge), silicon (Si), and tin (Sn); and
the Cr⁴⁺ being chromium substituted for less than five percent of N.

2. The nanophosphor of claim 1, the phosphor primarily having chemical formula Ca₂GeO₄:Cr⁴⁺.

3. The nanophosphor of claim 1, the phosphor primarily having chemical formula Ca₅Ge₃O₁₁:Cr⁴⁺.

4. The nanophosphor of claim 1, having particle size of less than 200 nanometers diameter.

5. The nanophosphor of claim 4, each particle having a silica coating.

6. The nanophosphor of claim 4, the silica coating having a thickness of 5-50 nm.

7. The nanophosphor of claim 5, comprising particles conjugated with polyethylene glycol and tissue selective compounds selected from the group consisting of antibodies, and nucleic acid chains.

8. The nanophosphor of claim 1, having particle size of less than 50 nanometers diameter.

9. The nanophosphor of claim 1, a surface of the nanophosphor being a hydrophilic surface.

10. A nanophosphor excitable by X-ray or blue-light radiation that, when excited, emits light in the near-infrared having wavelength between 1000-1700 nanometers comprising: phosphor having the chemical formula (M)ₓ(N)_{y}O_{z}:Cr⁴⁺;
the M being one or more elements selected from the group consisting of calcium (Ca), magnesium (Mg), strontium (Sr), barium (Ba), and zinc (Zn); and
the N being one or more elements selected from the group consisting of germanium (Ge), silicon (Si), and tin (Sn); and
the Cr⁴⁺ being chromium substituted for less than five percent of N.

11. The nanophosphor of claim 10, the phosphor primarily having chemical formula Ca₂GeO₄:Cr⁴⁺.

12. The nanophosphor of claim 10, the phosphor primarily having chemical formula Ca₅Ge₃O₁₁:Cr⁴⁺.

13. The nanophosphor of claim 10, having particle size of less than 200 nanometers diameter.

14. The nanophosphor of claim 13, each particle having a silica coating.

15. The nanophosphor of claim 14, the silica coating having a thickness of 5-50 nm.

16. The nanophosphor of claim 14, comprising particles conjugated with polyethylene glycol and tissue selective compounds selected from the group consisting of antibodies, and nucleic acid chains.

17. The nanophosphor of claim 10, having particle size of less than 50 nanometers diameter.

18. The nanophosphor of claim 10, a surface of the nanophosphor being a hydrophilic surface.
